(19) United States
(12) Patent Application Publication
(10) Pub. No.: US 2021/0137754 A1
(43) Pub. Date: May 13, 2021

(54) ABSORBENT PRODUCTS WITH ANTI-BACTERIAL AND ANTI-ODOUR PROPERTIES

(71) Applicant: Essity Hygiene and Health Aktiebolag, Goteborg (SE)

(72) Inventors: Cecile Sandin, Molndal (SE); Susanne Alenljung, Goteborg (SE)

(21) Appl. No.: 17/047,798

(22) PCT Filed: Apr. 27, 2018
(86) PCT No.: PCT/EP2018/060892

§ 371 Date: Oct. 15, 2020

(51) Int. Cl. A61F 13/84; A61F 13/47; A61F 13/496; A61F 13/49; A61L 15/60; A61L 15/44; A61L 15/34; A61L 15/24; A61L 15/20; A61L 15/18

(52) U.S. Cl. CPC A61F 13/8405; A61F 13/47; A61F 13/496; A61F 13/49; A61L 15/60; A61F 2013/530007; A61L 15/34; A61L 15/24; A61L 15/20; A61L 15/18; A61L 15/44

(57) ABSTRACT

A disposable absorbent hygiene product is provided, comprising an absorbent core disposed between a liquid pervious topsheet intended to face the wearer, and a backsheet intended to face away from the wearer, wherein said absorbent core comprises a super absorbent polymer composition that comprises a) super absorbent polymer particles including a cross-linked polymer of a water soluble ethylenically unsaturated monomer containing an acidic group, at least a part of said acidic groups being neutralized; and b) a particle size-controlled antibacterial agent that comprises a chelating agent containing EDTA or an alkali metal salt thereof; a mixture of an organic acid and a silicate-based salt; and a particle size control agent.

### CROSS-REFERENCE TO RELATED APPLICATION

The present application is a U.S. National Stage entry under 35 U.S.C. § 371 of, and claims priority to, International Application No. PCT/EP2018/060892, filed Apr. 27, 2018, the disclosure of which is hereby incorporated herein by reference in its entirety

### FIELD OF THE INVENTION

The present invention relates to a disposable absorbent hygiene product, comprising an absorbent core disposed between a liquid pervious topsheet intended to face the wearer, and a backsheet intended to face away from the wearer.

### BACKGROUND OF THE INVENTION

Disposable absorbent hygiene products are well known in the art and includes products worn in the urogenital area by users to absorb and store body exudates such as urine, faeces and menstrual fluids.

One important area of development in the area of absorbent products of the above-described type is the control of odorous compounds forming typically after the release of body fluids, especially over a longer period of time. These compounds include fatty acids, ammonia, amines, Sulphur-containing compounds and ketones and aldehydes. They are present as natural ingredients of body fluids or result from degradation processes of natural ingredients such as urea, which are frequently assisted by microorganisms occurring in the urogenital flora.

Various approaches exist to suppress the formation of unpleasant odors in absorbent articles, for instance the incorporation of odor inhibiting additives or deodorants such as zeolites and silica. The absorption of bodily liquids may however reduce the odor inhibiting capacity of zeolites as soon as these become saturated with water.

A second approach involves the addition of lactobacilli with the intention of inhibiting malodor-forming bacteria in the product.

Moreover, it is known that partially neutralized superabsorbent materials (acidic superabsorbent materials) counteract the formation of unpleasant odors in absorbent articles. However, the prior art acidic superabsorbent materials absorb lower amounts of body fluid compared to regular superabsorbent materials. In conventional attempts for introducing these various deodorizing/antibacterial functional components, even though the super absorbent polymer exhibits the deodorizing/antibacterial characteristics, dust formation occur during the process and thus processability is deteriorated, and there is a problem of deterioration in workability due to the dust formation. Further, in the case of the conventional method, there are disadvantages in that the stability of the super absorbent polymer is lowered and the functional ingredients themselves are expensive, and thus the unit price of the super absorbent polymer composition becomes high.

Accordingly, there has been a continuing demand for development of a super absorbent polymer composition that exhibits more improved antibacterial and deodorizing characteristics without deteriorating the basic absorption performance of the super absorbent polymer, and satisfies both stability and processability as well as economic efficiency.

### SUMMARY OF INVENTION

It is an object of the present invention to provide disposable absorbent hygiene products that can exhibit improved antibacterial and deodorizing characteristics.

The present inventors have found that the above-described object is at least partly met by a product in accordance with the appended claims.

The present disclosure therefore provides a disposable absorbent hygiene product, comprising an absorbent core disposed between a liquid pervious topsheet intended to face the wearer and a backsheet intended to face away from the wearer, wherein said absorbent core comprises a super absorbent polymer composition that comprises: super absorbent polymer particles including a cross-linked polymer of a water soluble ethylenically unsaturated monomer containing an acidic group, at least a part of said acidic groups being neutralized; and a particle size-controlled antibacterial agent that comprises a chelating agent containing EDTA or an alkali metal salt thereof; a mixture of an organic acid and a silicate-based salt; and a particle size control agent.

Using such super absorbent polymer composition in the absorbent core of a disposable absorbent hygiene product, highly improved antibacterial characteristics and associated deodorizing characteristics against bacteria inducing odor may be obtained, without deteriorating basic absorption performance, stability and processability.

### DETAILED DESCRIPTION OF THE INVENTION

Different aspects of the present disclosure will be described more fully hereinafter. The embodiments disclosed herein can, however, be realized in many different forms and the disclosure should not be construed as being limited to any particular embodiment, but includes all modifications, equivalents and alternatives falling within the spirit and scope of the disclosure.

The disposable absorbent hygiene product is intended to be worn with the liquid pervious topsheet towards the skin of the wearer, hereinafter also referred to as the body facing side of the product, and the backsheet facing away from the wearer, hereinafter also referred to as the garment facing side of the product.

The disposable absorbent hygiene product according to the present disclosure is intended to be worn in the urogenital area and may be absorbent product intended to be worn and held in place against the body by an undergarment, such an ordinary underwear or by specially adapted undergarments, such as a pad, for example an incontinence pad, a removable insert, or a sanitary napkin, or may be an absorbent product able to be worn and held against the body without external help from undergarment, such as an open diaper, a belt-type diaper or a pant diaper. The disposable absorbent hygiene product may be a unisex product, or may be specifically tailored to be used by men or females. The disposable absorbent hygiene product may be intended for children or adults.

In the context of the present disclosure, “disposable” is used in its ordinary sense to mean an article that is disposed or discarded after a limited number of usage events over varying lengths of time, for example less than about 10 events, less than about 5 events, or after 1 event.

A liquid permeable topsheet is arranged at the bodyfacing side of the disposable absorbent hygiene product. Materials suitable for topsheets are commonly known in the art of disposable absorbent hygiene products, and for the purposes of the present disclosure any material commonly known for use a topsheet materials may be used, including, but not limited to non-woven materials and perforated polymeric films.

The topsheet is suitably sufficiently fluid permeable to allow discharged body fluids such as urine to penetrate through the thickness of the topsheet. Also, the topsheet is suitably manufactured from a material which is compliant and soft-feeling to the skin of the wearer.

The topsheet may be manufactured from various web materials such as woven and nonwoven webs, perforated films, open cell foams, or combinations or laminates of the above-described materials.

In the context of the present disclosure, a “nonwoven” is a manufactured sheet, web or batt of directionally or randomly orientated fibers, bonded by friction, and/or cohesion and/or adhesion, excluding paper and products which are woven, knitted, tufted, stitch-bonded incorporating binding yarns or filaments, or felted by wet-milling, whether or not additionally needled. The fibers may be of natural or man-made origin and may be staple or continuous filaments or be formed in situ. Commercially available fibers have diameters ranging from less than about 0.001 mm to more than about 0.2 mm and they come in several different forms: short fibers (known as staple, or chopped), continuous single fibers (filaments or monofilaments), untwisted bundles of continuous filaments (tow), and twisted bundles of continuous filaments (yarn). Nonwoven fabrics can be formed by many processes such as meltblowing, spunbonding, solvent spinning, electrospinning, and carding.

The nonwoven materials to be used for the topsheet may, for example, be made of a spunbond, a spunbond/spunbond composite or a spunbond/meltblown composite, such as a SMS (spunbond/meltblown/spunbond), SSMS, SSMMS, SMMS, nonwoven material of polypropylene or bicomponent fibers of polypropylene and polyethylene, or of a combination of such materials. The topsheet may also have elastic properties.

The topsheet may be hydrophilized, hydrophilically treated, in order to improve the tendency for urine to penetrate the topsheet into the underlying structures. Methods for hydrophilizing nonwovens are known to those skilled in the art and include coating the nonwoven material with a hydrophilic coating, such as by applying a surfactant coating; by applying a hydrophilic monomer composition and a radical polymerization initiator onto the nonwoven followed by initiating a polymerization reaction on the nonwoven; by applying a coating of hydrophilic nanoparticles; or by treating the nonwoven surface with a high energy treatment (corona, plasma).

The topsheet material may have a basis weight of from 8 to 40, such as from 10 to 20, for example 12 to 17 g/m². However, the disclosure is not limited to topsheet materials having this basis weight only.

A backsheet is arranged at the garment facing side of the disposable absorbent hygiene product. Materials suitable as backsheets are commonly known in the art of disposable absorbent hygiene products. The backsheet prevents the exudates absorbed by the absorbent assembly from soiling other external articles that may contact the disposable absorbent hygiene product, such as bedsheets and undergarments. The backsheet may preferably be substantially impermeable to liquids, such as urine.

The backsheet may be substantially liquid impermeable but gas permeable, i.e. breathable, implying that air and other gases may pass through the backsheet, while being substantially impermeable to liquids. The Water Vapour Transmission Rate (WVTR) of the backsheet may, for example, be in the range of from 500 to 8000, such as from 1000 to 6000, for example from 2000 to 4000 g/m²/24 hours (as determined by ASTM E96)

For the purposes of the present disclosure, any material commonly known for use as backsheet materials may be included in the backsheet, including but not limited to polymeric films, for example films of polyethylene, polypropylene or copolymers of polyethylene or polypropylene, hydrophobized nonwoven materials, fluid impermeable foams and fluid impermeable laminates.

The backsheet may comprise one or more layers of material. For example, the backsheet may be a laminate of a liquid impermeable polymeric film towards the absorbent core and nonwoven towards the garment facing side, such as to provide a textile-like, soft feeling to the outer surface of the disposable absorbent hygiene product.

The disposable absorbent hygiene product according to the present disclosures comprises an absorbent core disposed between the topsheet and the backsheet, which absorbent core comprises a super absorbent polymer composition that comprises a super absorbent polymer particles including a cross-linked polymer of a water soluble ethylenically unsaturated monomer containing an acidic group, at least a part of said acidic groups being neutralized; and a particle size-controlled antibacterial agent that comprises a chelating agent containing EDTA or an alkali metal salt thereof; a mixture of an organic acid and a silicate-based salt; and a particle size control agent.

The purpose of the absorbent core is to absorb and retain body exudates, such as urine, faeces and/or menstrual fluid.

This super absorbent polymer composition will be described more in detail below.

The super absorbent polymer composition according to one embodiment of the present disclosure comprises:

a) absorbent polymer particle including a cross-linked polymer of a water-soluble ethylenically unsaturated monomer containing an acidic group, at least a part of the acidic group being neutralized; and

b) a particle size-controlled antibacterial agent including a chelating agent containing EDTA or an alkali metal salt thereof; a mixture of an organic acid and a silicate-based salt; and a particle size control agent.

First, the term “particle size-controlled agent” as used herein refers to an additive that functions to suppress the generation of dusts in the process by using the particle size control agent, in which in the particle size distribution of the antibacterial agent, the content ratio of the super absorbent polymer powder in the range of 150 to 850 μm is controlled upward as compared with a conventional case. That is, by using the particle size-controlled antibacterial agent, in the ratio distribution of a) powder having a particle size of 850 μm or more, b) a powder having a particle size of 600 to 850 μm, c) a powder having a particle size of 300 to 600 μm, d) a powder having a particle size of 150 to 300 μm, e) a powder having a particle size of from 45 to 150 μm, and f) a powder having a particle size of less than 45 μm, the content ratio of the super absorbent polymer powder having a size of 150 to 850 μm can be improved by 10 wt % or more and the content of the powder having a size of less than 150 μm can be reduced, compared to a conventional one.

The particle size-controlled antibacterial agent relates to a mixture of three components including a particle size control agent, a chelating agent containing EDTA or an alkali metal salt thereto, and a mixture of an organic acid and a silicate-based salt, which enable the super absorbent polymer composition to have antibacterial function.

By using a particle size control agent in an antibacterial agent comprising a chelating agent containing EDTA or an alkali metal salt thereof, and a mixture of an organic acid and a silicate-based salt, the super absorbent polymer composition can exhibit improved deodorizing/antibacterial characteristics compared with a conventional one. In particular, according to the results of experiments reported herein, it has been found that by adding to the super absorbent polymer particle a particle size-controlled antibacterial agent obtained by mixing the above three components, it is possible to very effectively suppress the growth of bacteria acting as malodorous components in disposable absorbent hygiene products, and at the same time remarkably reduce the amount of dusts generated during the application of the process. As a result, it was confirmed that both the workability and the processability can be improved without deteriorating the excellent antibacterial or deodorizing characteristics.

Moreover, when preparing a super absorbent polymer composition having an antibacterial activity, it is preferable that the content of the antibacterial agent is higher. However, when a substance other than the super absorbent polymer is added, it may cause deterioration of physical properties. According to the present disclosure, however, by using an appropriate amount of particle size control agent, excellent antibacterial efficiency can be exhibited and also dusts can be reduced. An antibacterial agent such as a chelating agent added for antibacterial activity can be a direct factor inducing fine particles. However, according to the present disclosure, by adding the particle size-controlled antibacterial agent, the amount of fine powders of the super absorbent polymer can be remarkably reduced relative to the conventional one, when compared based on the same amount of a conventional antibacterial agent.

Therefore, as the present antibacterial agent uses a particle size control agent in the mixture, it can reduce the amount of dust, indicating that the particle size is also controlled upward and the antibacterial agent mixture is not detached from SAP particles.

In addition, these components do not impair the stability and the like of the super absorbent polymer composition, so that the super absorbent polymer composition of the embodiment can maintain excellent basic absorption performance and its unit cost is also relatively low, which can greatly contribute to low unit price and high economic efficiency of the super absorbent polymer.

Therefore, the super absorbent polymer composition of one embodiment can be very usefully applied to various disposable absorbent hygiene products.

Hereinafter, the super absorbent polymer composition in accordance with the present disclosure will be described in more detail for each component.

The super absorbent polymer composition comprises a chelating agent including EDTA (ethylene diamine tetraacetic acid) or an alkali metal salt thereof and a mixture of an organic acid and a silicate-based salt for unique antibacterial/deodorizing effects. The alkalimetal salt of EDTA may, for example, be a sodium salt of EDTA which is EDTA-2Na or EDTA-4NaIn addition, or alternatively, to EDTA, amine acetic acid compounds such as selected from the group consisting of, cyclohexanediamine tetraacetic acid, diethylenetriamine pentaacetic acid, ethylene glycol-bis-(aminoethyl ether)-N,N,N′-triacetic acid, N-(2-hydroxyethyl)-ethylenediamine-N,N,N′-triacetic acid and triethylenetetraamine hexaacetic acid, and alkalimetal salts thereof, or various chelating agents may be used.

These chelating agents are present on the super absorbent polymer particles and thus may cause a synergistic effect with the mixture of the organic acid and the silicate-based salt. As a result, the super absorbent polymer composition can exhibit improved deodorizing/antibacterial characteristics.

More specifically, the chelating agent may act as an antibacterial agent, and thus have an antibacterial activity that inhibits the growth rates of various bacteria, in particular, the growth of Proteus mirabilis bacteria causing odor. However, despite the growth inhibitory action of the chelating agent, some bacteria remain, which causes malodors due to the generation of ammonia and the like. These malodors can be removed mainly by a mixture of an organic acid and a silicate-based salt, and as a result, the super absorbent polymer composition can exhibit excellent deodorizing/antibacterial characteristics due to the synergistic effect of the two components.

These chelating agents may be contained in an amount of 0.1 to 5 parts by weight, or 0.5 to 3 parts by weight, or 0.9 to 2 parts by weight based on 100 parts by weight of the super absorbent polymer particles. By using these chelating agents, they can suitably inhibit the growth rate of Proteus mirabilis bacteria which induce odor, to thereby exhibit excellent antibacterial characteristics, which can exhibit a preferable range of antibacterial activities (CFU/ml). Urea is changed into ammonia from Proteus mirabilis, and the amount of ammonia generated by inhibiting the growth of bacteria can be fundamentally controlled to be low. Therefore, the super absorbent polymer composition can exhibit excellent antibacterial/deodorizing characteristics. However, when the content of the chelating agent is excessively high, there is a possibility of killing even bacteria beneficial to a human body, or lowering the stability of the super absorbent polymer or deteriorating the absorption characteristics, which are not preferable.

Meanwhile, the super absorbent polymer composition also includes a mixture of an organic acid and a silicate-based salt. These organic acids and silicate salts may also be present on the super absorbent polymer particles.

These silicate salts can be in the form of a salt in which a silicate anion bonds ionically with a cation of an alkali metal or alkaline earth metal, and can exist in the state of particles. The particles of these silicate salts may include particles having a particle size between 150 μm or more and less than 600 μm in an amount of about 80 to about 98% by weight, or about 90 to about 99% by weight, or about 92 to about 99.3% by weight.

Moreover, the organic acid mixed with the silicate salt can exist on the super absorbent polymer particles in the form of particles having a particle size of 600 μm or less, or 150 μm to 600 μm.

Since the organic acid and the silicate may have the particle state and the particle size distribution as described above, they may be appropriately maintained on the super absorbent polymer particles to more selectively and efficiently adsorb the bacterial/malodorous components, thereby physically/chemically removing the components. As a result, the super absorbent polymer composition can exhibit more enhanced antibacterial/deodorizing characteristics. Furthermore, due to such particle state, it is also possible to exhibit anti-caking performance that does not induce caking when mixed with a super absorbent polymer.

The mixture of organic acid and silicate-based salt may include about 90 to 99.5% by weight, or about 95 to 99.3% by weight, or about 97 to 99.0% by weight of organic acid, based on the total weight of the mixture. Consequently, a large number of acid sites may be formed on the inside and/or the surface of the super absorbent polymer particles. When these acid sites are included, not only various malodorous components are physically adsorbed but also the hydrogen cation (H⁺) at the acid site is bonded to the malodorous components to form an ammonium salt, thereby more effectively removing the malodorous components.

The organic acid may include at least one selected from the group consisting of citric acid, fumaric acid, maleic acid, and lactic acid, but is not limited thereto.

The mixture of organic acid and silicate-based salt may be present in an amount of about 0.5 to about 5 parts by weight, or about 0.8 to about 5 parts by weight, or about 1 to about 4 parts by weight, based on 100 parts by weight of the super absorbent polymer. When the above component is contained in too small amount, the deodorizing characteristics due to the organic acid or the like may not be sufficient. When the above component is contained in too large amount, it is likely that the physical properties of the super absorbent polymer are deteriorated.

The mixture of the organic acid and the silicate-based salt may be prepared by a conventional method of mixing the organic acid and the silicate salt. Such a mixture may be prepared by previously mixing these two components, but these components may be separately mixed together with the chelating agent after preparation of the super absorbent polymer particles as described later.

Meanwhile, by using a particle size control agent after preparing an antibacterial agent exhibiting an antibacterial/deodorizing effect through mixing of the above three components, the particle size of the super absorbent polymer particles can be controlled, thereby remarkably reducing the generation of dusts during the preparation process of the super absorbent polymer.

In a conventional antibacterial agent having an antibacterial function, particles with a size distribution of #100 or less (150 μm or less), may, e.g., account for up to 18.35% by weight, and there arises a problem that a large amount of fine dust is generated when mixed with the super absorbent polymer. However, according to the present disclosure, as a particle size control agent is added to the chelating agent and the mixture of organic acid and silicate-based salt as described above, particles with a size distribution of #100 or less (150 μm or less) is not substantially present, or may be present at 0.5% by weight or less, preferably at 0.1% by weight or less. Therefore, not only the processability and workability can be improved, but also the content ratio of the super absorbent polymer powder having an average particle size of 150 to 850 um can be increased more than in the conventional one.

Thus, using the above results, in the case of the present invention, the particle size distribution range of 150 to 850 um can be controlled upward by the use of a particle size control agent.

In this case, the particle size control agent may be included in an amount of from 0.5 to 5 parts by weight based on 100 parts by weight of the total sum of the chelating agent and the mixture of organic acid and silicate-based salt. When the content of the particle size control agent is less than 0.5 part by weight, the effect of reducing dusts may be decreased , and when it exceeds 5 parts by weight, the physical properties may be deteriorated.

The particle size control agent may be at least one selected from the group consisting of mineral oil, natural oil, baby oil, corn oil, olive oil and silicone oil. According to a preferred embodiment, the particle size control agent may be mineral oil.

By using the particle size control agent in the antibacterial agent mixture, excellent antibacterial efficiency can be maintained, the particle size can be controlled upward, and the amount of dusts generated which determines whether the antibacterial agent mixture is detached from SAP particles can be reduced.

In addition, the particle size-controlled antibacterial agent may be contained in an amount of 0.1 to 5 parts by weight based on 100 parts by weight of the super absorbent polymer particles. Preferably, when the particle size-controlled antibacterial agent contains 1 to 4 parts by weight based on 100 parts by weight of the super absorbent polymer particles, the particle size distribution of #100 or less (or 150 μm or less) is controlled to be 0 to 1.5% by weight relative to the total weight, thereby more effectively reducing fine dusts, and the content ratio of the super absorbent polymer powder having a size of 150 to 850 um can be controlled upward as compared with a conventional one. If the content of the particle size-controlled antibacterial agent is less than 0.1 parts by weight, there is no antibacterial effect, and when it exceeds 5 parts by weight, deterioration of physical properties becomes severe.

Therefore, in the super absorbent polymer composition according to the present disclosure, the particle size-controlled antibacterial agent may be present on the super absorbent polymer particles.

Meanwhile, the type and the preparation method of the super absorbent polymer to be mixed with the particle size-controlled antibacterial agent which is the mixture of the three components described above are based on a method commonly used in the relevant technical field, and a step and method of mixing these components in the super absorbent polymer are not particularly limited.

For example, the super absorbent polymer can be obtained by carrying out a thermal polymerization or a photo-polymerization of a monomer composition including a water-soluble ethylenically unsaturated monomer and a polymerization initiator to obtain a hydrogel polymer, and subjecting the obtained hydrogel polymer to drying, pulverization, classification and the like. If necessary, the steps of surface cross-linking and reassembling fine particles can be further carried out.

For reference, the “super absorbent polymer” as used herein means a cross-linked polymer obtained by polymerizing a water-soluble ethylenically unsaturated monomer including an acidic group, at least a part of the acidic group being neutralized, or a base polymer prepared in the form of powder by drying and pulverizing the cross-linked polymer, or those prepared in a state suitable for commercialization by subjecting the cross-linked polymer or the base polymer to additional steps, for example, surface crosslinking, fine powder-reassembly, drying, pulverization, classification and the like.

As the water-soluble ethylenically unsaturated monomer, any monomer commonly used in the preparation of a super absorbent polymer can be used without particular limitation. Herein, any one or more monomers selected from the group consisting of an anionic monomer and a salt thereof, a nonionic hydrophilic group-containing monomer, and an amino group-containing unsaturated monomer and a quaternary product thereof may be used.

Specifically, the water-soluble ethylenically unsaturated monomer that can be used include any one or more selected from the group consisting of anionic monomers of acrylic acid, methacrylic acid, maleic anhydride, fumaric acid, crotonic acid, itaconic acid, 2-acryloylethane sulfonic acid, 2-methacryloylethane sulfonic acid, 2-(meth)acryloylpropane sulfonic acid, or 2-(meth)acrylamido-2-methylpropane sulfonic acid, and their salts; non-ionic, hydrophilic group-containing monomers of (meth)acrylamide, N-substituted (meth)acrylate, 2-hydroxyethyl(meth)acrylate, 2-hydroxypropyl(meth)acrylate, methoxypolyethylene glycol(meth)acrylate or polyethylene glycol (meth)acrylate; and amino group-containing unsaturated monomers of (N,N)-dimethylaminoethyl (meth)acrylate or (N,N)-dimethylaminopropyl (meth)acrylamide, and their quaternary product.

More preferably, acrylic acid or a salt thereof, for example, an alkali metal salt such as acrylic acid or a sodium salt thereof can be used. By using these monomers, it becomes possible to prepare a super absorbent polymer having more excellent physical properties. When the alkali metal salt of acrylic acid is used as the monomer, acrylic acid may be used by neutralizing it with a neutralizing agent such as sodium hydroxide (NaOH).

A polymerization initiator used in the polymerization of the water-soluble ethylenically unsaturated monomer is not particularly limited as long as it is generally used in the preparation of a super absorbent polymer.

Specifically, as the polymerization initiator, a thermal polymerization initiator, a photo-polymerization initiator by UV irradiation can be used depending on the polymerization method. However, even in the case of the photo-polymerization method, a certain amount of heat is generated by ultraviolet irradiation or the like, and a certain amount of heat is generated in accordance with the progress of the polymerization reaction, which is an exothermic reaction, and thus, a thermal polymerization initiator may further be included.

The photo-polymerization initiator that can be used is not particularly limited by its constitution as long as it is a compound capable of forming a radical by light such as ultraviolet rays.

The monomer composition may further include an internal cross-linking agent as a raw material of the super absorbent polymer. As the internal crosslinking agent, a crosslinking agent having at least one ethylenically unsaturated group while having at least one functional group capable of reacting with the water-soluble substituent of the water-soluble ethylenically unsaturated monomer; or a crosslinking agent having two or more functional groups capable of reacting with water-soluble substituents of the monomers and/or water-soluble substituents formed by hydrolysis of the monomers.

Specific examples of the internal crosslinking agent include bisacrylamide having 8 to 12 carbon atoms, bismethacrylamide having 8 to 12 carbon atoms, poly(meth)acrylate of polyol having 2 to 10 carbon atoms, poly(meth)allyl ether of polyol having 2 to 10 carbon atoms, or the like. More specifically, at least one selected from the group consisting of N,N′-methylenebis(meth)acrylate, ethyleneoxy(meth)acrylate, polyethyleneoxy(meth)acrylate, propyleneoxy(meth)acrylate, glycerin diacrylate, glycerin triacrylate, trimethylol triacrylate, triallylamine, triaryl cyanurate, triallyl isocyanate, polyethylene glycol, diethylene glycol, and propylene glycol can be used.

In the above-described preparation method, the monomer composition of the super absorbent polymer may further include additives such as a thickener, a plasticizer, a preservation stabilizer, an antioxidant and the like, if necessary.

The raw materials such as the above-described water-soluble ethylenically unsaturated monomers, photo-polymerization initiators, thermal polymerization initiators, internal cross-linking agents and additives can be prepared in the form of a monomer composition solution dissolved in a solvent.

Meanwhile, the method of forming a hydrogel polymer by thermal polymerization or photo-polymerization of such a monomer composition is not particularly limited by its construction as long as it is a polymerization method commonly used in the art.

Specifically, the polymerization method can be largely classified into a thermal polymerization and a photo-polymerization according to a polymerization energy source. Typically, in the case of the thermal polymerization, the polymerization may be carried out in a reactor like a kneader equipped with stirring spindles, and in the case of the photo-polymerization, the polymerization may be carried out in a reactor equipped with a movable conveyor belt, but the above-described polymerization method is only an example, and the present invention is not limited to the polymerization method described above.

In this case, the hydrogel polymer obtained by the above-described method may have generally a water content of about 40 to about 80% by weight. Meanwhile, the “water content” as used herein means a weight occupied by moisture with respect to a total amount of the hydrogel polymer, which may be the value obtained by subtracting the weight of the dried polymer from the weight of the hydrogel polymer. Specifically, the water content is defined as a value calculated by measuring the weight loss due to evaporation of moisture in the polymer in a process of raising and drying the temperature of the polymer through infrared heating. At this time, the water content is measured under the drying conditions determined as follows: the drying temperature is increased from room temperature to about 180° C. and then the temperature is maintained at 180° C., and the total drying time is set to 20 minutes, including 5 minutes for the temperature rising step.

Next, the hydrogel polymer thus obtained is dried.

At this time, a step of coarse pulverization may be further carried out before drying in order to improve the efficiency of the drying step, if necessary.

In this case, a pulverizing device used may include, but its configuration is not limited to, for example, any one pulverizing device selected from the group consisting of a vertical pulverizing device, a turbo cutter, a turbo grinder, a rotary cutter mill, a cutter mill, a disc mill, a shred crusher, a crusher, a chopper, and a disc cutter. However, it is not limited to the above-described examples.

In this case, the coarsely pulverizing step may be carried out so that the particle diameter of the hydrogel polymer becomes about 2 to about 10 mm.

The hydrogel polymer coarsely pulverized as described above or the hydrogel polymer immediately after polymerization without the coarsely pulverizing step is subjected to a drying step.

The drying method of the drying step may also be selected and used without being limited by its constitution as long as it is a method generally used for drying the hydrogel polymer. Specifically, the drying step may be carried out by a method such as hot air supply, infrared irradiation, microwave irradiation or ultraviolet irradiation. The water content of the polymer after such a drying step may be about 0.1% to about 10% by weight.

Next, the dried polymer obtained through such a drying step is pulverized.

The polymer powder obtained after the pulverizing step may have a particle diameter of about 150 to about 850 μm. Specific examples of a pulverizing device that can be used for pulverizing the polymer to have the above particle diameter may include a pin mill, a hammer mill, a screw mill, a roll mill, a disc mill, a jog mill or the like, but the present invention is not limited to the above-described examples.

Also, in order to control the physical properties of the super absorbent polymer powder finally commercialized after the pulverization step, a separate step of classifying the polymer powder obtained after the pulverization depending on the particle diameter may be undergone. Preferably, a polymer having a particle diameter of about 150 to about 850 μm is classified.

A step of surface cross-linking the pulverized or classified polymer can be further carried out. At this time, the surface crosslinking agent is not limited by its constitution as long as it is a compound capable of reacting with a functional group of the polymer. Examples of such a surface crosslinking agent include a polyhydric alcohol compound, a polyvalent alkylene carbonate compound, a polyvalent epoxy compound, or the like.

Further, the surface crosslinking agent may contain about 0.01 to about 5 parts by weight based on 100 parts by weight of the base polymer powder obtained from the pulverized or classified polymer.

Moreover, when the surface crosslinking agent is used, the surface crosslinking liquid may further contain water and/or methanol as a medium.

The surface cross-linking step may be carried out by heating at a temperature of 140 to 200° C. for 5 minutes to 80 minutes. Preferably, the base polymer powder to which the surface crosslinking liquid is added is subjected to a heat treatment at a maximum reaction temperature of 140° C. to 200° C., or 150° C. to 190° C. for 5 minutes to 80 minutes, or 10 minutes to 70 minutes, or 20 minutes to 65 minutes to carry out the surface cross-linking reaction. More specifically, in the surface cross-linking step, the heat treatment can be carried out by raising the temperature to the maximum reaction temperature over a period of 10 minutes to 40 minutes at an initial temperature of 20° C. to 130° C., or 40° C. to 120° C., and maintaining the maximum temperature for 5 minutes to 80 minutes.

The temperature raising means for the surface crosslinking reaction is not particularly limited. The heating can be carried out by providing a heating medium or directly providing a heating source. The type of heat medium that can be used here includes a heated fluid such as steam, hot air, hot oil, etc., but it is not limited thereto. Further, the temperature of the heating medium to be provided can be appropriately selected in consideration of the means of the heating medium, the temperature raising speed, and the temperature raising target temperature. Meanwhile, a heat source to be provided directly may include a heating method using electricity or a heating method using gas, but is not limited thereto.

Therefore, it is possible to provide a super absorbent polymer composition further comprising a surface cross-linked layer formed on the super absorbent polymer particles.

The super absorbent polymer composition according the present disclosure can be obtained by uniformly mixing the above-described super absorbent polymer particles obtained by the above process, the chelating agent, and the mixture of organic acid and silicate-based salt.

In this case, the mixing method is not particularly limited. For example, a method of adding a super absorbent polymer particle, a chelating agent, an organic acid and a silicate salt to a reaction tank and then mixing them, or spraying a solution containing a chelating agent, an organic acid and a silicate salt particle on the super absorbent polymer, a method of continuously supplying a super absorbent polymer, a chelating agent, an organic acid and a silicate salt particle in a reaction tank such as a continuously operated mixer and then mixing them, a method of previously mixing an organic acid and a silicate salt, and then continuously supplying and mixing a super absorbent polymer particle, a chelating agent, the previously mixed mixture of organic acid and silicate salt, or the like can be used.

Meanwhile, in the super absorbent polymer composition of the embodiment described above, the super absorbent polymer particles may further contain residual iron ions derived from the monomer composition containing the water-soluble ethylenically unsaturated monomer and/or the initiator in an amount of 3 ppm or less, or 0.1 to 3 ppm with respect to the total monomers.

A polymerization initiator such as a redox initiator is usually used during the preparation process of the super absorbent polymer particles, and the iron ions derived from these initiators may remain in the monomer and/or the super absorbent polymer particles. However, such iron ions may cause deterioration of the physical properties of the super absorbent polymer composition, and as the composition of one embodiment contains the chelating agent, the residual amount of the iron ions can be reduced. As a result, the super absorbent polymer composition of one embodiment can exhibit more excellent physical properties.

Meanwhile, the method for preparing a super absorbent polymer composition having antibacterial properties according to the present invention may comprise the steps of: mixing a chelating agent containing a certain amount of EDTA or an alkali metal salt thereof; a mixture of an organic acid and a silicate-based salt; and a particle size control agent to prepare a particle size-controlled antibacterial agent; and mixing a super absorbent polymer and the particle size-controlled antibacterial agent.

In this case, the constitution and conditions of the apparatus during preparation of the particle size-controlled antibacterial agent and the antibacterial super absorbent polymer particle are not particularly limited, and they can be prepared by stirring using a general mixer (for example, plowshare blender).

The super absorbent polymer composition according to one embodiment of the present invention as described above can exhibit excellent antibacterial/deodorizing effects and basic absorption characteristics.

Further, the final antibacterial super absorbent polymer composition to which the particle size-controlled antibacterial agent obtained by mixing the above-described four components has been added can become particles having an average particle diameter in the range of 150 to 850 μm. That is, in the present invention, by remarkably reducing the particle size of the fine particles among the particle size of the antibacterial agent, the amount of the fine particle of the entire super absorbent polymer composition can also be reduced, and in particular, the amount of distribution in particles in the range of 150 to 850 um can be increased. In particular, an antibacterial agent such as a chelating agent added for antibacterial activity can be a direct factor inducing fine particles. However, according to the present invention, by adding the particle size-controlled antibacterial agent, the amount of fine powder of the super absorbent polymer composition can be remarkably reduced relative to a conventional one, when compared based on the same amount of conventional antibacterial agent.

According to one embodiment, in the super absorbent polymer composition of the present invention, the content of the particles having an average particle diameter in the range of 150 to 850 μm obtained by classification in the content of the entire particle size distribution may be 99% by weight or more, preferably 99.1% by weight or more. At this time, since the super absorbent polymer composition basically contains a fine particle of the super absorbent polymer itself, the fine particle of the super absorbent polymer itself can also be included when including the antibacterial agent of the present invention. According to another embodiment, in the super absorbent polymer composition described above, the ratio of the super absorbent polymer powder having a particle size of less than 150 μm based on the amount of the entire particle size distribution is 1.5% by weight or less, the ratio of the absorbent polymer powder having a particle size of 850 μm or more may be 1% by weight or 0.8% by weight or less. In this case, in a ratio of the absorbent polymer powder having a particle size of less than 150 μm, the ratio of the absorbent polymer powder having an average particle size of less than 45 um can be 0.5% by weight or less, 0.05% by weight or less, or 0% by weight, and the ratio of the absorbent polymer powder having an average particle size of 45 to 150 μm can be 1% by weight or less, or 0.5% by weight or less. According to another aspect, based on the content of all the powders on the obtained particles in the average particle size distribution measured by using a standard sieve, a) the ratio of the powder having an average particle size of 850 μm or more may be 1% by weight or less, b) the ratio of the powder having a particle size of 600 to 850 μm may be 15 to 18% by weight, c) the ratio of the powder having a particle size of 300 to 600 μm may be 59 to 63% by weight, d) the ratio of the powder having a particle size of 150 to 300 μm may be 19 to 23% by weight, e) the ratio of the powder having a particle size of 45 to 150 μm may be 0.5% by weight or less, and f) the ratio of the powder having a particle size of less than 45 μm may be 0.5% by weight or less.

Different types of absorbent cores may be produced using the super absorbent polymer composition in accordance with the present disclosure, i.e., a super absorbent polymer composition comprising a) a super absorbent polymer particle including a cross-linked polymer of a water-soluble ethylenically unsaturated monomer containing an acidic group, at least a part of the acidic group being neutralized; and b) a particle size-controlled antibacterial agent including a chelating agent containing EDTA or an alkali metal salt thereof; a mixture of an organic acid and a silicate-based salt; and a particle size control agent.

The absorbent core may comprise from 10, from 15, from 20 or from 30, to 100, to 80, to 70, to 50, or to 40 wt % super absorbent polymer based on the total amount of absorbent material in the absorbent core. The super absorbent polymer in the absorbent core may be only consisted by the super absorbent polymer composition in accordance with the present disclosure, or may be a mixture of two or more types of super absorbent polymers, with the super absorbent polymer composition of the present disclosure constituting at least 10, at least 25, at least 50 or at least 75% by weight, based on the total amount of super absorbent polymer.

The super absorbent polymer composition according to the present disclosure may be used in absorbent cores comprising a mixture of mixture of cellulosic material and super absorbent polymers. Such cores are commonly known in the art and may in general be produced by different methods known to the person skilled in the art, such as by hammer milling fluff pulp, milled pulp with super absorbent polymer composition, depositing the mixture onto a core forming drum and debulking the core before transferring the drum to a substrate, such as a web material of the disposable absorbent hygiene product, for example to a topsheet material web or to a backsheet material web. Such cores are commonly known as airfelt-based cores.

Cellulosic materials that can be milled and then used in absorbent cores according to the present disclosure are well known in the art and include wood pulp, cotton, flax and peat moss. Wood pulp is preferred. Pulps can be obtained from mechanical or chemimechanical, sulfite, kraft, pulping reject materials, organic solvent pulps, etc. Both softwood and hardwood species are useful. Softwood pulps are preferred. It is not necessary to treat cellulosic fibers with chemical debonding agents, cross-linking agents and the like for use in the present material. Some portion of the pulp may be chemically treated for improved flexibility of the product. The flexibility of the material may also be improved by mechanically working the material or tenderizing the material.

An absorbent core based on a mixture of cellulosic fibres and super absorbent polymer may comprise from 10 to 80, such as from 15 to 70, for example from 20 to 50, or from 30 to 40 weight % of super absorbent polymer, based on the total weight of absorbent material in the absorbent core.

For the purposes herein, while the super absorbent polymer composition in accordance with the present disclosure comprises super absorbent polymer particles and a size-controlled anti-bacterial agent, such composition is nevertheless considered to represent super absorbent polymers, for example in calculations of concentrations of super absorbent polymers in a in an absorbent core.

The super absorbent polymer composition according to the present disclosure may be used in absorbent cores consisting of a laminate comprising a continuous or discontinuous layer of super absorbent polymer immobilized between two layers of thermoplastic material, of which at least one is liquid permeable, such as for instance between two nonwoven layers, or between a non-woven sheet and a network of fiberized hot-melt adhesive. Such a laminate may comprise at least 70, such as at least 80 or at least 90 wt % of super absorbent polymers, based on the total weight of absorbent material, or may be essentially cellulose-free, i.e. comprising essentially 100 wt % super absorbent polymer composition.

The disposable absorbent hygiene product according to the present disclosure may comprise one or more absorbent cores, all disposed between the topsheet and the backsheet, of which core(s) at least one, or all cores, comprises a super absorbent polymer composition comprising super absorbent polymer particles.

A disposable absorbent hygiene product may comprise multiple absorbent cores disposed in a stacked relationship, i.e. placed on top of each other such, such as with a first core being disposed between the topsheet and a second core, or in a side by side arrangement.

The different absorbent cores in a disposable absorbent hygiene product with multiple cores may have essentially the same or distinctly different composition, shape, basis weight, size, liquid retention capacity, thickness and/or basis weight.

For example, a disposable absorbent hygiene product may comprise a first, large absorbent core, for example, having a lower basis weight and a lower concentration (wt/wt) of super absorbent polymer and/or a larger planar surface area, and a second, smaller absorbent core, for example having a higher basis weight, a higher concentration (wt/wt) of super absorbent polymer and/or a smaller planar surface area, with the second core disposed between the topsheet and the first absorbent core or disposed between the backsheet and the first absorbent core.

The absorbent core may comprise the super absorbent polymer composition in accordance with the present disclosure alone, or may comprise a mixture with one or more different super absorbent polymers.

A disposable absorbent hygiene product in accordance with the present disclosure may comprise further components, for example additional components with the aim of providing odor preventing properties odor masking properties or anti-bacterial properties. Such may be introduced into the absorbent core, or may be provided on other material layers of an absorbent article, such as on the topsheet or on a liquid pervious layer between the topsheet and the absorbent core.

Examples of such additional components include lactic acid bacteria (see, e.g., EP 1 140 226), zeolites, active carbon (see, e.g., EP 2 916 880; WO2015/094068) cyclodextrins (see, e.g., EP 1 404 384), a combination of (i) an anti-bacterial agent selected from the group consisting of isothiazolinones and benzisothiazolinones, oxazolidines, pyridines, optionally chlorinated phenols, bromo compounds, aldehyde and dialdehyde compounds, benzyl alcohols, cresols, p-hydroxybenzoic acids and their esters and salts, organic acids and their alkali metal and earth alkaline metal salts, organic polyacids and their alkali metal and earth alkaline metal salts, and sulfites, bisulfites, nitrates, nitrites and iodates of alkali metals and earth alkaline metals, or at least one alkali metal or alkaline earth metal chloride with (ii) an organic zinc salt (see EP 2 083 873).

In addition to the topsheet, absorbent core(s) and backsheet, further components commonly employed in disposable absorbent hygiene products may also be employed in a disposable absorbent hygiene product according to the present disclosure.

An acquisition layer may be arranged between the topsheet and the absorbent core. While the absorbent core is intended to absorb and store body exudates, such as urine, it may be advantageous to include an acquisition layer between the topsheet and the absorbent assembly provide for interim acquisition of large amounts of liquid, as well as providing a layer for the distribution of liquid away from the immediate place of impact. Materials suitable as acquisition layers, also referred to in the art as transfer layer, or ADL (acquisition and distribution layer), are commonly known in the art of disposable absorbent hygiene products, and for the purposes of the present disclosure, any material known to the person skilled in the art as being useful as an acquisition layer may be used. An acquisition layer may, for example, be in the form of an airlaid layer, a spunlace layer, a high-loft, foam or any other type of material layer which may be used in an absorbent article to act as a liquid acquisition and absorption layer. The acquisition layer is suitably adapted to quickly receive and temporarily store discharged liquid before it is absorbed by the absorbent core. Such acquisition layer may be composed of, for example, airlaid nonwoven, spunlace nonwoven, high loft nonwoven or foam materials.

A wetness indicator, for example a material that changes its color upon contact with urine, may be included in the disposable absorbent hygiene product, such as disposed between the absorbent assembly and the backsheet and visible through the backsheet, such as to indicate whether a wetting event has taken place.

Moreover, when the disposable absorbent hygiene product is an incontinence pad or a panty liner, a fastening means, such as a strip of pressure sensitive adhesive, may be disposed on the garment facing side of the backsheet to provide secure placement of the pad in the underwear.

Hereinafter, the function and effect of the present invention will be described in more detail by way of specific examples of the present invention. It is to be understood, however, that these examples are provided for illustrative purposes only and the scope of the invention is not determined these examples.

### PREPARATION OF SUPER ABSORBENT POLYMER COMPOSITION

### Example 1

38.9 parts by weight of caustic soda (NaOH) and 103.9 parts by weight of water were mixed with 100 parts by weight of acrylic acid monomer, and 0.1 part by weight of sodium persulfate as a thermal polymerization initiator, 0.01 part by weight of diphenyl(2,4,6-trimethylbenzoyl)-phosphine oxide as a photo-polymerization initiator and 0.3 part by weight of polyethylene glycol diacrylate as a cross-linking agent were added to the above mixture to prepare a monomer composition.

The polymerization reaction of the monomer composition was carried out by irradiating ultraviolet rays for 1 minute while flowing at a flow rate of 243 kg/hr on a polymerization belt of a continuous type belt polymerization reactor in which an internal temperature is maintained at 80° C. and an ultraviolet irradiation device having an intensity of 10 mW as a mercury UV lamp light source is installed on the top, and the polymerization reaction was further continued for 2 minutes in a state of non-light source.

The gel type polymerization sheet which outputs after completion of polymerization was first cut using a shredder-type cutter and then coarsely crushed through a meat chopper. Thereafter, the resultant was dried at a temperature of 180° C. for 30 minutes through a hot-air drier, pulverized using a rotary mixer, and classified into 150 μm to 850 μm to prepare a base polymer.

0.1% by weight of ethylene glycol diglycidyl epoxide was added to the base polymer and uniformly mixed, and then a surface treatment reaction was carried out at 140° C. for 1 hour to obtain a super absorbent polymer.

Based on 100 parts by weight of the super absorbent polymer, i) 0.5 parts by weight of a sodium salt of EDTA (EDTA-2Na), ii) 2.02 parts by weight of a mixture in which 99% by weight of citric acid is mixed with 1% by weight of a sodium metasilicate salt, and iii) 0.25 parts by weight of mineral oil as a particle size control agent based on 100 parts by weight of the mixture of i) and ii) were added to a plowshare blender and stirred at 500 rpm for 5 minutes. The mixture of the three components thus prepared was referred to as a particle size-controlled antibacterial agent, and the particle size distribution of the antibacterial agent was as follows.

That is, based on the content of the entire powders on the obtained particles in the average particle size distribution measured by using a standard sieve, a) the ratio of the powder having an average particle size of 850 μm or more was 0.8% by weight, b) the ratio of the powder having a particle size of 600 to 850 μm was 16.6% by weight, c) the ratio of the powder having a particle size of 300 to 600 μm was 60.9% by weight, d) the ratio of the powder having a particle size of 150 to 300 μm was 21.6% by weight, e) the ratio of the powder having a particle size of 45 to 150 μm was 0.1% by weight, and f) the ratio of the powder having a particle size of less than 45 μm was 0% by weight.

### Devices and Reagents

Electronic scale (accuracy: 0.01 g), Sieve Shaker, Sieve (20, 30, 50, 100, 325 mesh standard sieve), Pan Receiver and Cap used, 250 ml Beaker

### Test Method

Pan Receiver was placed in the lowermost stage and stacked in order from the sieve with less meshes. 100 g of sample was quantitatively weighed into a 250 ml beaker and put in the uppermost stage sieve, and a lid was closed. This was fixed to a sieve shaker and shaken for 10 minutes. After shaking for 10 minutes, the sample remaining in each sieve wire net was collected and precisely weighted. At this time, care was taken so that the sample did not detach from the outside, and the measurement amplitude was set to 1.0 mm.

### Calculation Method

The amount remaining in each sieve was calculated by Equation 1 below.

Amount remaining in each sieve (%)=(Weight of sample remaining in each sieve)/(Weight of entire sample)×100 [Equation 1]

### Record

20 mesh or more-particle, 20 to 30 mesh-particle, 30 to 50 mesh-particle, 50 to 100 mesh-particle, 100 to 325 mesh-particle, and less than 325 mesh-particle were measured, respectively.

At this time, the particle size was given to two decimal places, and the particle size of “less than 325 mesh” was rounded off to be a significant figure and recorded in Data Sheet.

Then, 100 parts by weight of the super absorbent polymer and 2.52 parts by weight of the particle size-controlled antimicrobial agent were mixed, and the super absorbent resin composition thus prepared was used as Example 1. In addition, based on the total amount of the super absorbent polymer composition (which means super absorbent polymer+particle size-controlled antibacterial agent), the ratio of the super absorbent polymer particles in the range of 150 μm to 850 μm was 97% by weight or more, the ratio of the super absorbent polymer particles in the range of 45 to 150 μm was 1.5% by weight or less, the ratio of the super absorbent polymer particles of less than 45 um was 0% by weight or less, and the ratio of the super absorbent polymer particles of 850 μm or more was 1.0% by weight.

In the components of the antimicrobial agent, when the content of EDTA-2Na was low to be 0.5 parts by weight and 0.8 parts by weight, it exhibited only the tendency of dusts according to the increase in the content of mineral oil, and the antibacterial efficiency was measured when the content of EDTA-2Na was 1.0 parts by weight. Further, the PSD of the final antibacterial SAP was measured only at this time.

### Example 2

A super absorbent polymer was prepared in the same manner as in Example 1.

Based on 100 parts by weight of the super absorbent polymer, i) 0.5 parts by weight of a sodium salt of EDTA (EDTA-2Na), ii) 2.02 parts by weight of a mixture in which 99% by weight of citric acid was mixed with 1% by weight of a sodium metasilicate salt, and iii) 0.5 part by weight of mineral oil as a particle size control agent based on 100 parts by weight of the mixture of i) and ii) were added to a plowshare blender and stirred at 500 rpm for 5 minutes. The mixture of the three components thus prepared was referred to as a particle size-controlled antibacterial agent, and the particle size distribution ratio of the antibacterial agent was the same as that of Example 1.

Then, 100 parts by weight of the super absorbent polymer and 2.52 parts by weight of the particle size-controlled antimicrobial agent were mixed, and the super absorbent resin composition thus prepared was used as Example 2.

### Example 3

A super absorbent polymer was prepared in the same manner as in Example 1.

Based on 100 parts by weight of the super absorbent polymer, i) 0.5 parts by weight of a sodium salt of EDTA (EDTA-2Na), ii) 2.02 parts by weight of a mixture in which 99% by weight of citric acid was mixed with 1% by weight of a sodium metasilicate salt, and iii) 1 part by weight of mineral oil as a particle size control agent based on 100 parts by weight of the mixture of i) and ii) were added to a plowshare blender and stirred at 500 rpm for 5 minutes. The mixture of the three components thus prepared was referred to as a particle size-controlled antibacterial agent. The particle size distribution ratio of the antibacterial agent was the same as that of Example 1.

Then, 100 parts by weight of the super absorbent polymer and 2.52 parts by weight of the particle size-controlled antimicrobial agent were mixed, and the super absorbent resin composition thus prepared was used as Example 3.

### Example 4

A super absorbent polymer was prepared in the same manner as in Example 1.

Based on 100 parts by weight of the super absorbent polymer, i) 0.5 parts by weight of a sodium salt of EDTA (EDTA-2Na), ii) 2.02 parts by weight of a mixture in which 99% by weight of citric acid was mixed with 1% by weight of a sodium metasilicate salt, and iii) 1.5 part by weight of mineral oil as a particle size control agent based on 100 parts by weight of the mixture of i) and ii) were added to a plowshare blender and stirred at 500 rpm for 5 minutes. The mixture of the three components thus prepared was referred to as a particle size-controlled antibacterial agent. The particle size distribution ratio of the antibacterial agent was the same as that of Example 1.

Then, 100 parts by weight of the super absorbent polymer and 2.52 parts by weight of the particle size-controlled antimicrobial agent were mixed, and the super absorbent resin composition thus prepared was used as Example 4.

### Example 5

A super absorbent polymer was prepared in the same manner as in Example 1.

Based on 100 parts by weight of the super absorbent polymer, i) 0.8 parts by weight of a sodium salt of EDTA (EDTA-2Na), ii) 2.02 parts by weight of a mixture in which 99% by weight of citric acid was mixed with 1% by weight of a sodium metasilicate salt, and iii) 0.5 part by weight of mineral oil as a particle size control agent based on 100 parts by weight of the mixture of i) and ii) were added to a plowshare blender and stirred at 500 rpm for 5 minutes. The mixture of the three components thus prepared was referred to as a particle size-controlled antibacterial agent. The particle size distribution ratio of the antibacterial agent was the same as that of Example 1.

Then, 100 parts by weight of the super absorbent polymer and 2.82 parts by weight of the particle size-controlled antimicrobial agent were mixed, and the super absorbent resin composition thus prepared was used as Example 5.

### Example 6

A super absorbent polymer was prepared in the same manner as in Example 1.

Based on 100 parts by weight of the super absorbent polymer, i) 0.8 parts by weight of a sodium salt of EDTA (EDTA-2Na), ii) 2.02 parts by weight of a mixture in which 99% by weight of citric acid was mixed with 1% by weight of a sodium metasilicate salt, and iii) 1 part by weight of mineral oil as a particle size control agent based on 100 parts by weight of the mixture of i) and ii) were added to a plowshare blender and stirred at 500 rpm for 5 minutes. The mixture of the three components thus prepared was referred to as a particle size-controlled antibacterial agent. The particle size distribution ratio of the antibacterial agent was the same as that of Example 1.

Then, 100 parts by weight of the super absorbent polymer and 2.82 parts by weight of the particle size-controlled antimicrobial agent were mixed, and the super absorbent resin composition thus prepared was used as Example 6.

### Example 7

A super absorbent polymer was prepared in the same manner as in Example 1.

Based on 100 parts by weight of the super absorbent polymer, i) 0.8 parts by weight of a sodium salt of EDTA (EDTA-2Na), ii) 2.02 parts by weight of a mixture in which 99% by weight of citric acid was mixed with 1% by weight of a sodium metasilicate salt, and iii) 1.5 part by weight of mineral oil as a particle size control agent based on 100 parts by weight of the mixture of i) and ii) were added to a plowshare blender and stirred at 500 rpm for 5 minutes. The mixture of the three components thus prepared was referred to as a particle size-controlled antibacterial agent. The particle size distribution ratio of the antibacterial agent was the same as that of Example 1.

Then, 100 parts by weight of the super absorbent polymer and 2.82 parts by weight of the particle size-controlled antimicrobial agent were mixed, and the super absorbent resin thus prepared composition was used as Example 7.

### Example 8

A super absorbent polymer was prepared in the same manner as in Example 1.

Based on 100 parts by weight of the super absorbent polymer, i) 0.8 parts by weight of a sodium salt of EDTA (EDTA-2Na), ii) 2.02 parts by weight of a mixture in which 99% by weight of citric acid was mixed with 1% by weight of a sodium metasilicate salt, and iii) 2 parts by weight of mineral oil as a particle size control agent based on 100 parts by weight of the mixture of i) and ii) were added to a plowshare blender and stirred at 500 rpm for 5 minutes. The mixture of the three components thus prepared was referred to as a particle size-controlled antibacterial agent. The particle size distribution ratio of the antibacterial agent was the same as that of Example 1.

Then, 100 parts by weight of the super absorbent polymer and 2.82 parts by weight of the particle size-controlled antimicrobial agent were mixed, and the super absorbent resin composition thus prepared was used as Example 8.

### Example 9

A super absorbent polymer was prepared in the same manner as in Example 1.

Based on 100 parts by weight of the super absorbent polymer, i) 1 part by weight of a sodium salt of EDTA (EDTA-2Na), ii) 2.02 parts by weight of a mixture in which 99% by weight of citric acid was mixed with 1% by weight of a sodium metasilicate salt, and iii) 0.125 parts by weight of mineral oil as a particle size control agent based on 100 parts by weight of the mixture of i) and ii) were added to a plowshare blender and stirred at 500 rpm for 5 minutes. The mixture of the three components thus prepared was referred to as a particle size-controlled antibacterial agent. The particle size distribution ratio of the antibacterial agent was the same as that of Example 1.

Then, 100 parts by weight of the super absorbent polymer and 3.02 parts by weight of the particle size-controlled antimicrobial agent were mixed, and the super absorbent resin composition thus prepared was used as Example 9.

### Example 10

A super absorbent polymer was prepared in the same manner as in Example 1.

Based on 100 parts by weight of the super absorbent polymer, i) 1 part by weight of a sodium salt of EDTA (EDTA-2Na), ii) 2.02 parts by weight of a mixture in which 99% by weight of citric acid was mixed with 1% by weight of a sodium metasilicate salt, and iii) 0.25 parts by weight of mineral oil as a particle size control agent based on 100 parts by weight of the mixture of i) and ii) were added to a plowshare blender and stirred at 500 rpm for 5 minutes. The mixture of the three components thus prepared was referred to as a particle size-controlled antibacterial agent. The particle size distribution ratio of the antibacterial agent was the same as that of Example 1.

Then, 100 parts by weight of the super absorbent polymer and 3.02 parts by weight of the particle size-controlled antimicrobial agent were mixed, and the super absorbent resin composition thus prepared was used as Example 10.

### Example 11

A super absorbent polymer was prepared in the same manner as in Example 1.

Based on 100 parts by weight of the super absorbent polymer, i) 1 part by weight of a sodium salt of EDTA (EDTA-2Na), ii) 2.02 parts by weight of a mixture in which 99% by weight of citric acid was mixed with 1% by weight of a sodium metasilicate salt, and iii) 0.5 parts by weight of mineral oil as a particle size control agent based on 100 parts by weight of the mixture of i) and ii) were added to a plowshare blender and stirred at 500 rpm for 5 minutes. The mixture of the three components thus prepared was referred to as a particle size-controlled antibacterial agent. The particle size distribution ratio of the antibacterial agent was the same as that of Example 1.

Then, 100 parts by weight of the super absorbent polymer and 3.02 parts by weight of the particle size-controlled antimicrobial agent were mixed, and the super absorbent resin composition thus prepared was used as Example 11.

### Example 12

A super absorbent polymer was prepared in the same manner as in Example 1.

Based on 100 parts by weight of the super absorbent polymer, i) 1 part by weight of a sodium salt of EDTA (EDTA-2Na), ii) 2.02 parts by weight of a mixture in which 99% by weight of citric acid was mixed with 1% by weight of a sodium metasilicate salt, and iii) 1 part by weight of mineral oil as a particle size control agent based on 100 parts by weight of the mixture of i) and ii) were added to a plowshare blender and stirred at 500 rpm for 5 minutes. The mixture of the three components thus prepared was referred to as a particle size-controlled antibacterial agent. The particle size distribution ratio of the antibacterial agent was the same as that of Example 1.

Then, 100 parts by weight of the super absorbent polymer and 3.02 parts by weight of the particle size-controlled antimicrobial agent were mixed, and the super absorbent resin thus prepared composition was used as Example 12.

### Example 13

A super absorbent polymer was prepared in the same manner as in Example 1.

Based on 100 parts by weight of the super absorbent polymer, i) 1 part by weight of a sodium salt of EDTA (EDTA-2Na), ii) 2.02 parts by weight of a mixture in which 99% by weight of citric acid was mixed with 1% by weight of a sodium metasilicate salt, and iii) 2 parts by weight of mineral oil as a particle size control agent based on 100 parts by weight of the mixture of i) and ii) were added to a plowshare blender and stirred at 500 rpm for 5 minutes. The mixture of the three components thus prepared was referred to as a particle size-controlled antibacterial agent. The particle size distribution ratio of the antibacterial agent was the same as that of Example 1.

Then, 100 parts by weight of the super absorbent polymer and 3.02 parts by weight of the particle size-controlled antimicrobial agent were mixed, and the super absorbent resin composition thus prepared was used as Example 13.

Based on the total amount of the super absorbent polymer composition in which the particle size-controlled antimicrobial agent was used (which means super absorbent polymer particle size-controlled antibacterial agent), the ratio of the super absorbent polymer particles in the range of 150 μm to 850 μm was 97% by weight or more, the ratio of the super absorbent polymer particles between 45 or more and less than 150 μm was 1.5% by weight or less, the ratio of the super absorbent polymer particles of less 45 um was 0% by weight, and the ratio of the super absorbent polymer particles of 850 μm or more was 1% by weight.

### Comparative Example 1

A super absorbent polymer was prepared in the same manner as in Example 1.

Based on 100 parts by weight of the super absorbent polymer, i) 0.25 part by weight of a sodium salt of EDTA (EDTA-2Na), and ii) 2.02 parts by weight of a mixture in which 99% by weight of citric acid was mixed with 1% by weight of a sodium metasilicate salt were added to a plowshare blender and stirred at 500 rpm for 2 minutes.

Then, 100 parts by weight of the super absorbent polymer and 2.27 parts by weight of the mixture of the two components previously prepared were mixed, and the super absorbent resin composition thus prepared was used as Comparative Example 1.

The particle size distribution of the antibacterial agent mixture in which the mineral oil used here was not used is the same as in Comparative Example 1.

In the particle size distribution of the antimicrobial agent mixture in which the mineral oil used here was not used, the ratio of the super absorbent polymer particles in the range of 150 μm to 850 μm was 82% by weight, the ratio of the super absorbent polymer particles in the range of 45 μm to 150 μm was 13 to 15% by weight, the ratio of the super absorbent polymer particles in the range of less than 45 um is 4 to 6% by weight, and the ratio of the super absorbent polymer particles in the range of 850 μm or more was 0.5% by weight or less.

### Comparative Example 2

A super absorbent polymer was prepared in the same manner as in Example 1.

Based on 100 parts by weight of the super absorbent polymer, i) 0.5 part by weight of a sodium salt of EDTA (EDTA-2Na), and ii) 2.02 parts by weight of a mixture in which 99% by weight of citric acid was mixed with 1% by weight of a sodium metasilicate salt were added to a plowshare blender and stirred at 500 rpm for 2 minutes.

Then, 100 parts by weight of the super absorbent polymer and 2.52 parts by weight of the mixture of the two components previously prepared were mixed, and the super absorbent resin composition thus prepared was used as Comparative Example 2.

The particle size distribution of the antibacterial agent mixture in which the mineral oil used here was not used is the same as in Comparative Example 1.

### Comparative Example 3

A super absorbent polymer was prepared in the same manner as in Example 1.

Based on 100 parts by weight of the super absorbent polymer, i) 1 part by weight of a sodium salt of EDTA (EDTA-2Na), and ii) 2.02 parts by weight of a mixture in which 99% by weight of citric acid was mixed with 1% by weight of a sodium metasilicate salt were added to a plowshare blender and stirred at 500 rpm for 2 minutes.

Then, 100 parts by weight of the super absorbent polymer and 3.02 parts by weight of the mixture of the two components previously prepared were mixed, and the super absorbent resin composition thus prepared was used as Comparative Example 3.

The particle size distribution of the antibacterial agent mixture in which the mineral oil used here was not used is the same as in Comparative Example 1.

Based on the total amount of the super absorbent polymer composition in which the particle size-uncontrolled antimicrobial agent was used (which means super absorbent polymer+particle size-uncontrolled antibacterial agent), a ratio of the super absorbent polymer particles in the range of 150 μm to 850 μm was 97% by weight or more, a ratio of the super absorbent polymer particles of 45 to 150 μm was 1.5 to 3% by weight, a ratio of the super absorbent polymer particles of less 45 um was 0.2 to 1.0% by weight, and a ratio of the super absorbent polymer particles of 850 μm or more was 0.5% by weight or less.

### Reference Example 1

i) 1 part by weight of a sodium salt of EDTA (EDTA-2Na), and ii) 2.02 parts by weight of a mixture in which 99% by weight of citric acid was mixed with 1% by weight of a sodium metasilicate salt were added to a plowshare blender and stirred at 500 rpm for 2 minutes. The mixture thus prepared was used as Reference Example 1.

### Reference Example 2

i) 0.5 part by weight of a sodium salt of EDTA (EDTA-2Na), and ii) 2.02 parts by weight of a mixture in which 99% by weight of citric acid was mixed with 1% by weight of a sodium metasilicate salt were added to a plowshare blender and stirred at 500 rpm for 2 minutes. The mixture thus prepared was used as Reference Example 2.

In the average particle size distribution measured by using a standard sieve, all the antimicrobial agent mixtures of Reference Examples 1 and 2 showed that a) a ratio of a powder having a particle size of 850 μm or more is 0.5% by weight or less, b) the ratio of a powder having a particle size of 600 to 850 um was 13 to 16% by weight, c) a ratio of a powder having a particle size of 300 to 600 um was 35 to 38% by weight, d) a ratio of a powder having a particle size of 150 to 300 um was 26 to 29% by weight, e) a ratio of a powder having a particle size of 45 to 150 um was 12 to 14% by weight, and f) a ratio of a powder having a particle size of less than 45 um was 4 to 6% by weight

### Evaluation of Physical Properties of Super Absorbent Resin

Physical properties of the super absorbent polymer compositions of Examples 1 to 13 and Comparative Examples 1 to 3 were measured by the following method, and the results are shown in Tables 1 and 2 below.

### (1) Antibacterial/Deodorizing Performance Test

50 ml of artificial urine inoculated with Proteus mirabillis (ATCC 29906) at 250,000 CFU/ml was cultured in an oven at 35° C. for 12 hours. After incubation with this artificial urine for 12 hours, the obtained artificial urine was used as a control group, which was thoroughly washed with 150 ml of brine to measure CFU (Colony Forming Unit), and thereby the physical properties of the control group were calculated.

2 g of the super absorbent polymers and the super absorbent polymer compositions of Examples 9 to 13 and Comparative Examples 1 to 3 were added to 50 ml of artificial urine inoculated with Proteus mirabillis (ATCC 29906) at 250,000 CFU/ml and shaken for 1 minute, allowing it to uniformly mix. Thereafter, it was incubated in an oven at 35° C. for 12 hours. Artificial urine after incubation for 12 hours was thoroughly washed with 150 ml of saline to measure CFU (Colony Forming Unit). Thus, the antibacterial/deodorizing characteristics of the respective Examples and Comparative Examples were calculated/evaluated.

### (2) Measurement of DUST Number

The DUST number was analyzed using Dustview II (Palas GmbH) capable of measuring the level of dust of the super absorbent polymer by laser.

The dust number was measured using 30 g of the SAP sample prepared in Examples or Comparative Examples. Since the small particles and the specific substances have fallen at a slower rate than the coarse particles, the dust number was calculated by the following Equation 2.

Dust number=Max value+30 sec.valu [Equation 2]

(in Equation 2, the Max value represents the maximum dust number, and the 30 sec. value is a value measured after 30 seconds after it reached the maximum dust number.)

### (3) Flowability

The super absorbent polymers prepared in Examples or Comparative Examples were thoroughly mixed so that the particle size could be uniformly mixed, 100±0.5 g of each sample was taken and poured into a 250 ml beaker. After placing the density measuring cup in the middle under the funnel with the lowest part diameter of 1 cm (unit), the funnel hole was closed and the weighted sample was lightly poured and filled in the funnel. At the moment of opening the hole of the funnel that was closed, a stopwatch was operated to measure the time (in seconds) required until all of the samples have completely fallen to the lowest part of the funnel. All procedures were conducted in a constant temperature and humidity chamber (temperature 23±2° C., relative humidity 45±10%).

### (4) Bulk Density

100 g of each super absorbent polymer was flowed through an orifice of a standard fluidity measuring device and received in a 100 ml container, and was cut out so that the super absorbent polymer was horizontal. After adjusting the volume of the super absorbent polymer to 100 ml, the weight of only the super absorbent polymer excluding the container was measured. Then, the bulk density corresponding to the weight of the super absorbent polymer per unit volume was obtained by dividing the weight of the super absorbent polymer by 100 ml which is the volume of the super absorbent polymer.

### (5) Centrifuge Retention Capacity (CRC)

The centrifuge retention capacity (CRC) for a physiological saline solution by absorption magnification under no load was measured in accordance with EDANA (European Disposables and Nonwovens Association) recommended test method No. WSP 241.3. W₀(g) (about 0.2 g) of the super absorbent polymer was uniformly put in a nonwoven fabric-made bag, followed by sealing. Then, the bag was immersed in a physiological saline solution composed of 0.9 wt % aqueous sodium chloride solution at room temperature. After 30 minutes, water was removed from the bag by centrifugation at 250 G for 3 minutes, and the weight W₂(g) of the bag was then measured. Further, the same procedure was carried out without using the super absorbent polymer, and then the resultant weight W₁(g) was measured. Using the respective weights thus obtained, CRC (g/g) was determined according to the following Equation 3.

CRC(g/g)={[W₂(g)−W₁(g)−W₀(g)]/W₀(g)} [Equation 3]

TABLE 1

| | Content of | | | | |
|---|---|---|---|---|---|
| | EDTA-2Na in | | | | |
| | antibacterial | Particle size | | Bulk | |
| | functional agent | control agent | Flowability | density | Dust |
| | (parts by weight) | (part by weight) | (sec) | (g/cm³) | number |
| Reference Example 1 | 1 | 0 | 7.3 | 0.84 | 18.4 |
| (Antibacterial agent alone) | | | | | |
| Reference Example 2 | 0.5 | 0 | 6.2 | 0.89 | 20.8 |
| (Antibacterial agent alone) | | | | | |
| GS401N | — | — | 9.2 | 0.62 | 1.1 |
| Comparative Example 1 | 0.25 | 0 | 9.0 | 0.69 | 2.4 |
| Comparative Example 2 | 0.5 | 0 | 9.0 | 0.69 | 3.2 |
| Comparative Example 3 | 1.0 | 0 | 8.9 | 0.70 | 6.1 |
| Example 1 | 0.5 | 0.25 | 9.0 | 0.69 | 2.6 |
| Example 2 | | 0.5 | 9.0 | 0.69 | 1.9 |
| Example 3 | | 1.0 | 9.1 | 0.68 | 1.4 |
| Example 4 | | 1.5 | 9.3 | 0.68 | 1.3 |
| Example 5 | 0.8 | 0.5 | 9.1 | 0.69 | 1.9 |
| Example 6 | | 1.0 | 9.3 | 0.69 | 1.9 |
| Example 7 | | 1.5 | 9.3 | 0.67 | 1.4 |
| Example 8 | | 2.0 | 9.4 | 0.68 | 0.6 |
| Example 9 | 1.0 | 0.125 | 9.5 | 0.68 | 3.5 |
| Example 10 | | 0.25 | 9.4 | 0.68 | 3.2 |
| Example 11 | | 0.5 | 9.3 | 0.69 | 2.7 |
| Example 12 | | 1 | 9.5 | 0.68 | 2.1 |
| Example 13 | | 2 | 9.8 | 0.65 | 1.0 |

TABLE 2

| | | Measurement of | | |
|---|---|---|---|---|
| | Culture | antibacterial efficiency | | CRC |
| | time | CFU/ml | log[CFU/ml] | (g/g) |
| Control group | 0 h | 250000 | 5.40 | — |
| | after 12 h | 110,000,000 | 8.04 | — |
| Super absorbent | after 12 h | 1,200,000 | 6.08 | 37.3 |
| polymer alone | | | | |
| Comparative | after 12 h | 880,000 | 5.94 | 36.6 |
| Example 1 | | | | |
| Comparative | after 12 h | 350,000 | 5.54 | 36.3 |
| Example 2 | | | | |
| Comparative | after 12 h | 210,000 | 5.32 | 36.0 |
| Example 3 | | | | |
| Example 9 | after 12 h | 270,000 | 5.43 | 35.5 |
| Example 10 | after 12 h | 260,000 | 5.41 | 35.6 |
| Example 11 | after 12 h | 360,000 | 5.56 | 34.8 |
| Example 12 | after 12 h | 300,000 | 5.48 | 34.5 |
| Example 13 | after 12 h | 240,000 | 5.38 | 35.8 |

Referring to Tables 1 and 2, it is confirmed that in the case of the super absorbent polymer compositions of Examples, by adding a specific amount of the particle size control agent to the functional additives, the antibacterial efficiency is maintained and enhanced antibacterial/deodorizing characteristics are exhibited while maintaining at least the same level of centrifuge retention capacity relative to Comparative Examples. In particular, it can be seen that Examples of the present invention can provide an antibacterial super absorbent polymer composition which remarkably reduces the dust number generated during the process relative to Comparative Examples, thereby satisfying both stability and processability.

At this time, since the direct factor that induces dust in the antimicrobial agent mixture is EDTA-2Na, the higher the content of EDTA-2Na causes dust problem. Thus, in the case of Comparative Examples 1 to 3, there arises a problem that the dust number increases when the content of EDTA-2Na increases.

Meanwhile, in the case of Examples 1 to 13 of the present invention, as a certain amount of the particle size control agent is added even when the content of EDTA-2Na increases, the dust number could be relatively more reduced than in Comparative Examples 1 to 3. In addition, the antibacterial efficiency of the super absorbent polymer using the antimicrobial agent described in the present invention remained excellent compared with the case of the super absorbent polymer alone.

What is claimed is:

1. A disposable absorbent hygiene product, comprising an absorbent core disposed between a liquid pervious topsheet intended to face the wearer, and a backsheet intended to face away from the wearer, wherein said absorbent core comprises a super absorbent polymer composition that comprises:
a. super absorbent polymer particles including a cross-linked polymer of a water soluble ethylenically unsaturated monomer containing an acidic group, at least a part of said acidic groups being neutralized; and
b. a particle size-controlled antibacterial agent that comprises
i. a chelating agent containing EDTA or an alkali metal salt thereof;
ii. a mixture of an organic acid and a silicate-based salt; and
iii. a particle size control agent.

2. The disposable absorbent hygiene product according to claim 1, wherein said super absorbent polymer composition is contained in said absorbent core in an amount of from 10 to 100 parts by weight, based on 100 parts by weight of absorbent material in the absorbent core.

3. The disposable absorbent hygiene product according to claim 1, wherein said absorbent core comprises a mixture of said super absorbent polymer composition and milled fluff pulp.

4. The disposable absorbent hygiene product according to claim 3, wherein said super absorbent polymer composition is contained in said absorbent core in an amount of from 10 to 80 parts by weight, based on 100 parts by weight of absorbent material in the absorbent core.

5. A disposable absorbent hygiene product according to claim 1, wherein said absorbent core comprises a laminate comprising a layer of said super absorbent polymer composition disposed between two layers of thermoplastic material, of which layers at least one is liquid permeable.

6. The disposable absorbent hygiene product according to claim 5, wherein said super absorbent polymer composition is contained in said absorbent core in an amount of from 70 to 100 parts by weight, based on 100 parts by weight of absorbent material in the absorbent core.

7. A disposable absorbent hygiene product according to claim 1, said product being an open diaper, a pant diaper, a belted type diaper, an incontinence pad or a sanitary napkin.

8. A disposable absorbent hygiene product according to claim 1, wherein said backsheet is substantially liquid impermeable.

9. The disposable absorbent hygiene product according to claim 1, wherein the particle size control agent is contained in an amount of 0.1 to 5 parts by weight based on 100 parts by weight of the total sum of the chelating agent and the mixture of organic acid and silicate-based salt.

10. The disposable absorbent hygiene product according to claim 1, wherein the particle size control agent is at least one selected from the group consisting of mineral oil, natural oil, baby oil, corn oil, olive oil and silicone oil.

11. The disposable absorbent hygiene product according to claim 1, wherein the particle size-controlled antibacterial agent is contained in an amount of 0.1 to 5 parts by weight based on 100 parts by weight of the super absorbent polymer particles.

12. The disposable absorbent hygiene product according to claim 1, wherein the chelating agent further includes at least one selected from the group consisting of cyclohexanediamine tetraacetic acid, diethylenetriamine pentaacetic acid, ethylene glycol-bis-(aminoethylether)-N,N,N′-triacetic acid, N-(2-hydroxyethyl)-ethylenediamine-N,N,N′-triacetic acid and triethylenetetraamine hexaacetic acid, or an alkali metal salt thereof.

13. The disposable absorbent hygiene product according to claim 1, wherein the chelating agent includes a sodium salt of EDTA, such as EDTA-2Na or EDTA-4Na.

14. The disposable absorbent hygiene product according to claim 1, wherein the chelating agents is contained in an amount of 0.1 to 5 parts by weight based on 100 parts by weight of the super absorbent polymer particles.

15. The disposable absorbent hygiene product according to claim 1, wherein the organic acid includes at least one selected from the group consisting of citric acid, fumaric acid, maleic acid, and lactic acid.

16. The disposable absorbent hygiene product according to claim 1, wherein the silicate-based salt includes a salt in which a silicate anion bonds ionically with a cation of an alkali metal or alkaline earth metal.

17. The disposable absorbent hygiene product according to claim 1, wherein organic acid includes 90 to 99.5% by weight based on the total weight of the mixture of organic acid and silicate-based salt.

18. The disposable absorbent hygiene product according to claim 1, wherein the mixture of organic acid and silicate-based salt is contained in an amount of 0.5 to 5 parts by weight based on 100 parts by weight of the super absorbent polymer particle.

19. The disposable absorbent hygiene product according to claim 1, wherein the water-soluble ethylenically unsaturated monomer includes at least one selected from the group consisting of anionic monomers of acrylic acid, methacrylic acid, maleic anhydride, fumaric acid, crotonic acid, itaconic acid, 2-acryloylethanesulfonic acid, 2-methacryloylethanesulfonic acid, 2-(meth)acryloylpropanesulfonic acid, or 2-(meth)acrylamido-2-methylpropanesulfonic acid, and their salts; non-ionic, hydrophilic group-containing monomers of (meth)acrylamide, N-substituted (meth)acrylate, 2-hydroxyethyl(meth)acrylate, 2-hydroxypropyl(meth)acrylate, methoxypolyethylene glycol(meth)acrylate or polyethylene glycol (meth)acrylate; and amino group-containing unsaturated monomers of (N,N)-dimethylaminoethyl (meth)acrylate or (N,N)-dimethylaminopropyl (meth)acrylamide, and their quaternary product.

20. The disposable absorbent hygiene product according to claim 1, further comprising a surface cross-linked layer formed on the super absorbent polymer particles.

21. The disposable absorbent hygiene product according to claim 1, wherein in the super absorbent polymer composition, the proportion of particles having an average particle diameter of less than 150 μm is at most 1.5 wt %.

22. The disposable absorbent hygiene product according to claim 1, wherein in the super absorbent polymer composition, the proportion of particles having an average particle diameter of less than 45 μm is at most 0.5 wt %.

23. The disposable absorbent hygiene product according to claim 1, wherein in the super absorbent polymer composition, the proportion of particles having an average particle diameter of above 850 μm is at most 1 wt %.

24. The disposable absorbent hygiene product according to claim 1, wherein in the super absorbent polymer composition, the content of particles having an average particle diameter in the range of 150 to 850 μm is at least 99 weight %.
